(19) United States
(12) Patent Application Publication
(10) Pub. No.: US 2012/0269751 A1
(43) Pub. Date: Oct. 25, 2012

(54) COMPOSITION FOR TOPICAL APPLICATION, USES THEREOF, APPLICATOR DEVICE AND KIT OF PARTS

(75) Inventor: Robert Sebastian Stal, Amsterdam (NL)

(73) Assignee: Medner Holding BV, Amsterdam (NL)

(21) Appl. No.: 13/509,447

(22) PCT Filed: Nov. 11, 2010
(86) PCT No.: PCT/NL10/50750

§ 371 Date: Jul. 6, 2012

(30) Foreign Application Priority Data
Nov. 11, 2009 (NL) 2003786

(51) Int. Cl. A61K 8/36; A61P 31/10; A61Q 3/00

(52) U.S. Cl. 424/61

(57) ABSTRACT

A composition for topical application includes at least one physiologically acceptable acid capable of reducing the pH of the skin of a treated person to the range of 1.5-4.5, and a physiologically acceptable carrier. Various uses for the treatment of skin and nail conditions are also disclosed, including and especially focusing on microbiological infections of the nail (onychomycosis). An applicator device and kit of parts comprising such a composition is also disclosed.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention relates to a composition for topical application. The invention further relates to various uses for the treatment of skin and nail conditions, including and especially focussing on microbiological infections of the nail (onychomycosis). The invention further relates to an applicator device and kit of parts comprising such a composition.

2. Description of Related Art Including Information Disclosed Under 37 CFR 1.97 and 37 CFR 1.98.

Onychomycosis is a wide-spread microbiological infection of the keratin of the nail. The fungi are able to enter the nail through microscopic cracks in the nail. Once in the nail the fungus uses the macroscopic molecules in the nail to feed itself and alter the environment to its own advantage. One of these alterations includes raising the pH of the nail to an alkaline level. This creates a favourable environment for the fungus in which it can multiply sexually instead of asexually.

### BRIEF SUMMARY OF THE INVENTION

It is an object of the invention to provide a safe and effective treatment for onychomycosis.

The invention relates to a composition for topical application, comprising at least one physiologically acceptable acid capable of reducing the pH of the skin and/or nails of a treated person to the range of 1.5-4.5, and a physiologically acceptable carrier. Bringing the pH into the range of 1.5-4.5 hampers the development of onychomycosis. The acid can be incorporated in for instance a cream of lotion. The physiologically acceptable carrier should be selected for not neutralising the effect of the acid.

Preferably, the physiologically acceptable acid is a carboxylic acid. Carboxylic acids are capable of achieving the desired pH drop on the infected skin and/or nails, and are typically well tolerated by the treated person. Preferably, the carboxylic acid is an alpha-hydroxy carboxylic acid.

Most preferably, the physiologically acceptable acid is a C1-C6 carboxylic acid.

In a preferred embodiment, the carboxylic acid is elected from the group consisting of lactic acid, malic acid, tartaric acid, citric acid, acetic acid, proprionic acid, isoproprionic acid, oxalic acid, glutaric acid, adipic acid, glycolic acid and mandelic acid.

Most preferably, the carboxylic acid is lactic acid. Lactic acid gives the desired result against onychomycosis and is well tolerated by persons.

Another preferred carboxylic acid is acetic acid. Acetic acid gives good results against onchyomycosis, is well tolerated. As acetic acid is a relatively cheap compound, in particular compared to lactic acid, acetic acid is a good alternative for lactic acid, and may also be used in an acetic acid/lactic acid combination in order to diminish the amount of lactic acid used while retaining the desired skin or nail acidifying effect.

It is preferred if the carboxylic acid is present in a quantity of at least 1% by weight. In this quantity, the development onychomycosis is effectively hampered.

Composition according to any of the preceding claims, wherein the composition comprises at least 10% by weight of lactic acid. In this quantity, the development onychomycosis is severely hindered and in some cases even stopped.

Preferably, the composition also comprises at least one C1-C4 alkyl ester of the carboxylic acid in a molecular ratio free acid to the ester of at least 1:100, preferably at least 1:20, most preferably at least 1:10. Such C1-C4 alkyl esters are excellent carriers for the acid.

In a preferred embodiment, the composition comprises lactic acid as the physiologically acceptable acid, and lactic acid ethyl ester as a physiologically acceptable carrier.

In another preferred embodiment, the composition comprises acetic acid as the physiologically acceptable acid, and lactic acid ethyl ester as a physiologically acceptable carrier.

It is also possible to obtain a satisfactory composition by mixing lactic acid and acetic acid as physiologically acceptable acids, using lactic acid ethyl ester as a physiologically acceptable carrier.

The invention relates to the use of a composition according to the invention for the preparation of a product for the treatment of skin and/or nail conditions.

The invention also relates to the use of a composition according to the invention for the preparation of a product for the treatment of nail conditions.

The invention further relates to the use of a composition according to the invention for the preparation of a product for the treatment of onychomycosis.

The invention moreover relates to the use of a composition according to any of the preceding claims 1-8 for the preparation of a product for the treatment of topical microbial infections.

The invention also relates to the use of a composition capable of lowering the pH of the human skin and/or nails to the range of 1.5-4.5 for the preparation of a product for the treatment of skin and/or nail conditions.

The invention relates to a device, comprising a container comprising a composition according to the invention, and an applicator connected to the container, wherein the applicator is adapted to apply the composition from the container to a part of human skin and/or nails to be treated.

The invention further relates to a kit of parts, comprising at least one container comprising a composition according to the invention and at least one scrub device for scrubbing a skin and/or nails part treated with the composition.

The invention provides a composition for topical application, including an effective amount of lactic acid, and/or a physiologically acceptable ester or salt thereof. The combination of lactic acid, or a similar acid, or a derivative thereof and at least one physiologically acceptable carrier to aid penetration into the nail were shown to yield an improved effect in the treatment of skin and nail conditions, in particular the treatment of onychomycosis. It is postulated that nail conditions benefit from the ability of lactic acid in controlling the pH of the nail, an essential part of this formula to combat nail fungus. This is enhanced by presence of the C1-C4 alkyl ester of a carboxylic acid as a carrier and stabilizer, which improves the penetration of lactic acid or its derivatives into the skin or nail.

The lactic acid is preferably mixed homogeneously with the C1-C4 alkyl ester of a carboxylic acid. Most preferably, the lactic acid is dissolved in the C1-C4 alkyl ester of a carboxylic acid. The C1-C4 alkyl ester of a carboxylic acid is preferably present as a major component of the composition, in a quantity of at least 50% by weight of the composition, preferably at least 90%.

The composition may include additional suitable components, for instance fragrances, emulsifiers, detergents, antioxidants and preservatives, and other ingredients commonly used in pharmaceutical and cosmetic formulations. Preferably, the composition is essentially free of water, which increases the stability of the composition over time. Preferably, the composition is formulated as a fluid composition such as a cream, or more preferably as a liquid composition such as a tonic, which is relatively easy to apply to the human skin and/or nails.

The C1-C4 esters include methyl, ethyl, n-propyl, isopropyl, n-butyl, isobutyl, sec-butyl and tert-butyl esters. Ethyl esters are preferred. In case the carboxylic acid contains multiple carboxyl-groups, at least one of the carboxyl groups is a C1-C4 ester. In case at least two carboxyl groups are esterified, the C1-C4 esters of these groups may be the same or different. Preferably, the C1-C4 alkyl ester is derived from a physiologically acceptable alpha-hydroxy carboxylic acid.

Preferably, the C1-C4 alkyl ester is derived from a physiologically acceptable carboxylic acid selected from the group consisting of lactic acid, malic acid, tartaric acid, citric acid, acetic acid, proprionic acid, isoproprionic acid, oxalic acid, glutaric acid, adipic acid, glycolic acid and mandelic acid. The C1-C4 esters of these acids are particularly effective. The ethyl esters of these compounds are preferred. C1-C4 alkyl esters of lactic acid was shown to be the most versatile of these compounds, and is therefore the most preferred.

In a preferred embodiment, the C1-C4 alkyl ester is lactic acid ethyl ester. This compound showed the best results.

Instead of lactic acid as the free acid, also lactic acid salt derivatives or esters may be used. Good results are obtained when the composition comprises at least one lactic acid derivative selected from the group consisting of sodium salicylate, potassium salicylate, calcium salicylate, magnesium salicylate, bismuth subsalicylate, monoethylammonium (MEA) salicylate, triethylammonium (TEA) salicylate, sulfolactic acid, ethyl salicylate, butyloctyl salicylate, C12-15 Alkyl salicylate, capryolyl lactic acid, hexyldecyl salicylate, isocetyl salicylate, isodecyl salicylate, ethylhexyl salicylate, methyl salicylate, myristyl salicylate, and thdecyl salicylate. Instead of lactic acid, an equivalent dose of a derivative having the same effect may be used. For salicylate salts, this is usually an equimolar amount. A composition may comprise a mix of lactic acid and/or one or more lactic acid derivatives.

In a preferred embodiment, the composition comprises at least 0.1% by weight of lactic acid or a physiologically acceptable ester or salt thereof, preferably between 0.5 and 30% by weight. Such amounts have an advantageous effect on various skin and/or nail conditions, in particular onychomycosis. The amount may be optimized for a specific skin and/or nail condition.

It is preferred if the composition also comprises the free acid or salt derived from the carboxylic acid used in the C1-C4 alkyl ester in a molecular ratio of at least 1:100 with respect to the C1-C4 alkyl ester, preferably at least 1:20, most preferably at least 1:10. Thus, a more stable composition is achieved. The availability of the carboxylic acid prevents the reversal of the esterification reaction in the presence of water, resulting in the free carboxylic acid and alcohol. For instance, a composition according to the invention, based on lactic acid ethyl ester, could be stabilized by adding lactic acid in a ratio of at least 1:100, preferably at least 1:20, most preferably at least 1:10

In a preferred embodiment, the composition comprises at least 50% by weight C1-C4 lactic acid ethyl ester, lactic acid in a ratio of at least 1:100 with respect to lactic acid ethyl ester, and at least 0.1% by weight of lactic acid, or a physiologically acceptable ester of salt thereof.

Preferably, the composition also comprises a nail-penetrating agent. A nail penetrating agent allows the acid and optional other active ingredients to permeate into the nail, which was found to lead to a better inhibition of the growth of nail infections, in particular fungal nail infections.

Preferred nail-penetrating agents are selected from the group consisting of urea, dimethyl isosorbide and ethyl lactate, C1-C4 alkyl ester of the carboxylic acid, preferably ethyl lactate or ethyl acetate. Mixtures of various nail penetrating agents may be used advantageously.

The invention relates to the use of a composition according to the invention for the preparation of a product, for instance a medicament, for the treatment of skin and nail conditions. Skin conditions the composition according to the invention may be used against conditions including onychomycosis and other microbiological infections. Typically, the amount of lactic acid or derivatives thereof are within 0.1-30% by weight.

The invention also relates to a device, comprising a container comprising a composition according to the invention, and an applicator connected to the container, wherein the applicator is adapted to apply the composition from the container to a part of human skin to be treated. Thus, the composition according to the invention is easy to apply and store.

The invention further relates to a kit of parts, comprising at least one container comprising a composition according to the invention and at least one scrub device for scrubbing a skin part treated with the composition, optionally also comprising a skin cream. Thus, skin conditions can be treated with a composition according to the invention and subsequently scrubbed mechanically by the scrub device.

### BRIEF DESCRIPTION OF THE DRAWINGS

FIG. 1 shows an applicator for applying a composition according to the invention.

### DETAILED DESCRIPTION OF THE INVENTION

The invention will now be further elucidated by the following non-limiting examples.

Applicator Device

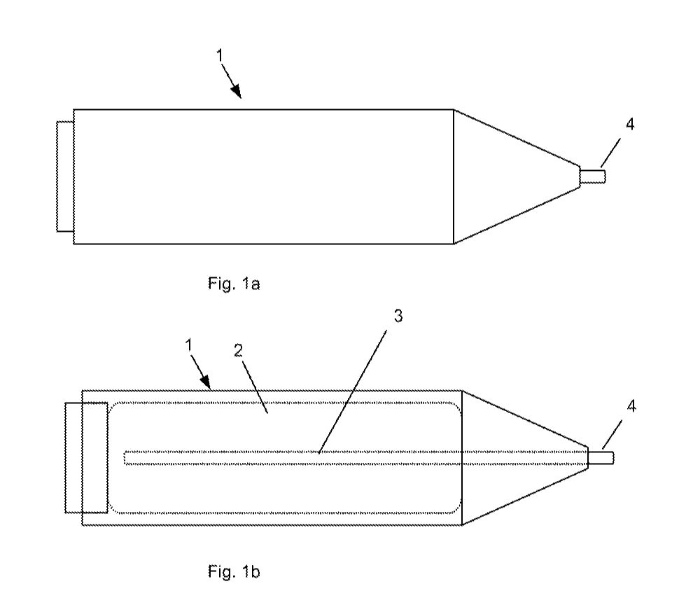

FIG. 1 shows an application device, somewhat resembling a felt-tipped type marker. The device comprises a reservoir containing a composition according to the invention. The device is pencil-shaped, and suitable to be hand-held. The reservoir is provided with an absorbing element made of a liquid-absorbing material capable of capillary action. This absorbing element dips into the liquid composition and extends from the inner part reservoir to the outside of the reservoir. The liquid contained in the reservoir within the marker and would be applied via a tip connecting to or made out of the distal end of the absorbing element extending out of the reservoir. Due to capillary action, the tip remains moist with the liquid product. For such an applicator device, the viscosity of 0 the liquid composition will have to be sufficiently low. The device may be provided with a cap to prevent volatile solvents of the composition to evaporate and dry the tip out. The moist tip is contacted with the skin part to be treated, preferably while applying some pressure, in order to apply the composition from the reservoir.

The reservoir may be filled with different preferred embodiments according to the invention, depending on the envisaged skin condition to be treated. For all of these applications it was shown that the combination of lactic acid dissolved in or mixed with at least one physiologically acceptable C1-C4 alkyl ester of a carboxylic acid as a carrier yielded a better absorbance of lactic acid into the skin than a comparable aqueous composition with the same lactic acid concentration.

All components are commercially available; the compositions were prepared using well-known mixing and blending techniques. pH measurements on the skin can be determined using commercially available pH meters, for instance the pH meter ‘1140’ from Mettler Toledo.

### Example 1

### Onychomycosis

An effective composition for treatment of onychomycosis was prepared by well know mixing techniques (percentages by weight):
lactic acid 5%
lactic acid ethyl ester 95%

The resulting liquid is applied as a tonic, and showed a changed environment of the nail to the fungus disadvantage. The pH on the skin of treated persons temporarily dropped to a value in the range of 2-4. Such low pH environments appear to hamper the development of onychomycosis. Over time this effectively treated the nail of nail fungus.

### Example 2

### Onychomycosis

An effective composition for treatment of onychomycosis was prepared by well know mixing techniques (percentages by weight):
Melaleuca Alternifolia 1%
Lavendula Officinalis 2%
Callitris Intratropica 2%
lactic acid 2%
lactic acid ethyl ester 93%

The resulting liquid is applied as an tonic, and showed a changed the environment of the nail to the fungus disadvantage, showing a temporary pH drop of the skin to approximately 3-4. Over time this effectively treated the nail of nail fungus. This example is cosmetically more attractive than example 1.

### Example 3

### Microbial Infections of the Skin

An effective composition against warts was prepared by well know mixing techniques (percentages by weight):
lactic acid 20%
lactic acid ethyl ester 80%

Upon application to the skin, the pH of the skin dropped to approximately 2.

Various types of microbial infections were successfully treated using this composition, resulting in complete or partial removal of the infection after multiple treatments.

### Example 4

### Onychomycosis

An effective composition for treatment of onychomycosis was prepared by well know mixing techniques (percentages by weight):
acetic acid 5%
ethyl acid ethyl ester 95%

The resulting liquid is applied as a tonic, and showed a changed environment of the nail to the fungus disadvantage. The pH on the nail of treated persons temporarily dropped to a value in the range of 2-4. Such low pH environments appear to hamper the development of onychomycosis. Over time this effectively treated the nail of nail fungus. The same composition using a mixture of 1% lactic acid and 4% acetic acid instead of 5% acetic acid also yielded satisfactory results against onchyomycosis.

### Example 5

### Onychomycosis

An effective composition for treatment of onychomycosis was prepared by well know mixing techniques (percentages by weight):
lactic acid 15%
urea 5%
lactic acid ethyl ester 80%

The resulting liquid is applied as an tonic, and showed a changed the environment of the nail to the fungus disadvantage, showing a temporary pH drop of the nail to approximately 3-4. Over time this effectively treated the nail of nail fungus. This 5 example is cosmetically more attractive than example 1.

### Example 6

### Microbial Infections of the Nail

An effective composition against nail microbial infections was prepared by well know mixing techniques (percentages by weight):
acetic acid 15%
dimethyl isosorbide 10%
acetic acid ethyl ester 75%

Upon application to the skin, the pH of the nail dropped to approximately 2.5. Dimethyl isosorbide acts as a nail-penetrating agent, enhancing the penetration by acetic acid. Various types of microbial infections were successfully treated using this composition, resulting in complete or partial removal of the infection after multiple treatments.

The compositions shown in the examples above may be used as such, or may be processed further to a final product, for instance by diluting with water or other solvents, or incorporation into a cream or lotion.

1. Composition for the treatment of fungal infections, in particular nail infections, comprising
at least one physiologically acceptable acid capable of reducing the pH in keratinous tissue, in particular nails, of a treated person below 4.5, preferably in the range of 1.5-4.5, and
a physiologically acceptable carrier.

2. Composition according to claim 1, wherein the physiologically acceptable acid is a carboxylic acid.

3. Composition according to claim 1 or 2, wherein the physiologically acceptable acid is a C1-C6 carboxylic acid

4. Composition according to claim 2, wherein the carboxylic acid is elected from the group consisting of lactic acid, malic acid, tartaric acid, citric acid, acetic acid, propionic acid, isopropionic acid, oxalic acid, glutaric acid, adipic acid, and glycolic acid.

5. Composition according to claim 4, wherein the carboxylic acid is lactic acid.

6. Composition according to claim 4, wherein the carboxylic acid is acetic acid.

7. Composition according to any of the preceding claims, wherein the composition comprises at least 10% by weight of lactic acid and/or acetic acid.

8. Composition according to any of the preceding claims 2-7, wherein the composition also comprises at least one C1-C4 alkyl ester of the carboxylic acid in a molecular ratio free acid to the alkyl ester of at least 1:100, preferably at least 1:20, most preferably at least 1:10.

9. Composition according to any of the preceding claims, wherein the composition also comprises a nail-penetrating agent.

10. Composition according to claim 9, wherein the nail-penetrating agent is selected from the group consisting of urea, dimethyl isosorbide and ethyl lactate, C1-C4 alkyl ester of the carboxylic acid, preferably ethyl lactate or ethyl acetate.

11. Composition according to any of the preceding claims, wherein the composition is essentially free of essential oils.

12. Use of a composition according to any of the preceding claims for the preparation of a product for the treatment of fungal infections in keratinous tissue, in particular nail infections, more in particular superficial, cutaneous and/or subcutaneous mycosis, more in particular onychomycosis or tinea.

13. Method of lowering the pH of a human nail below 4.5 for the prevention and/or retention of the development of fungal infections in keratinous tissue, in particular superficial, cutaneous and/or subcutaneous mycosis, more in particular onychomycosis or tinea.

14. Use of an acidic agent for lowering the pH of a nail having a fungal nail condition, in particular superficial, cutaneous and/or subcutaneous mycosis, more in particular onychomycosis or tinea.

15. Use according to claim 14, wherein the acidic agent comprises lactic acid, acetic acid or a mixture thereof.
